(19) United States
(12) Patent Application Publication
(10) Pub. No.: US 2009/0149338 A1
(43) Pub. Date: Jun. 11, 2009

(54) SYSTEM FOR DETECTING PROTEIN-PROTEIN INTERACTIONS

(76) Inventors: Thomas E. Hughes, Bozeman, MT (US); Robert G. Mealer, Bozeman, MT (US); Catherine H. Berlot, Sunbury, PA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/088,595

(22) PCT Filed: Sep. 29, 2006
(86) PCT No.: PCT/US2006/037933

§ 371 Date: Sep. 8, 2008

Related U.S. Application Data
(60) Provisional application No. 60/722,764, filed on Sep. 30, 2005.

(51) Int. Cl. C40B 30/04; C12Q 1/68; C12N 15/11; C07K 14/00

(52) U.S. Cl. 506/9; 435/6; 536/23.4; 530/350

(57) ABSTRACT

A method of detecting protein interactions is described wherein reporter protein fragments are genetically fused at internal positions of suspected interacting proteins. When proteins interact, the fluorescent fragments are brought close enough together to form a functional reporter protein providing visible confirmation of interaction.

### CROSS-REFERENCE TO RELATED APPLICATIONS

This application claims benefit of priority to U.S. provisional application 60/722,764, filed Sep. 30, 2005, which is herein incorporated by reference in its entirety.

### FIELD OF INVENTION

This invention relates to the use of complementation assays for the study of protein interactions.

### BACKGROUND OF THE INVENTION

Every process within a cell involves highly orchestrated interactions between different proteins. Determining which proteins interact, where they interact, and when they interact is critical to understanding most cellular processes. To date, the best strategy for identifying and characterizing protein interactions is complementation. There are now several complementation systems and most involve bringing two different protein domains together to produce a functional signal. The best example is the two hybrid approach in yeast where a DNA binding domain and a transcription activator have to be brought together by fusion protein partners for biological activity (Fields and Song, 1989, Nature, 340:245-246; U.S. Pat. No. 5,283,173 by Fields and Song). The yeast two hybrid approach is widely used throughout academia and industry, but it does have some drawbacks. For example, the fusion partners, the interacting proteins, must be in the nucleus to produce transcription. Accordingly, proteins that do not end up in the nucleus do not produce a signal in this system. Although other yeast two hybrid systems have since been developed that allow screening of cytoplasmic interactions, these systems are still in yeast and do not provide temporal/spatial resolution of fluorescence in live mammalian cells.

A recent improvement was the discovery that the Green Fluorescent Protein (GFP), or mutant forms of GFP, can be fragmented to create a complementation assay (US Patent Application Publication No. 2004/0235064 A1). Here, the two fragments of the fluorescent protein are attached to the ends of two other proteins. A fluorescent protein will be produced only if the two fusion partners interact. This has the advantage of working with proteins that exist outside the nucleus, and it works in the context of many different expression systems, including mammalian cells. The limitation to this approach is that the ends of the interacting proteins have to be close enough to one another to form the fluorescent protein.

The Green Fluorescent Protein

The GFP excitation spectrum shows an absorption band (blue light) maximally at 395 nm with a minor peak at 470 nm, and an emission peak (green light) at 509 nm. The longer-wavelength excitation peak has greater photostability then the shorter peak, but is relatively low in amplitude (Chalfie et al., 1994, Science, 263:802-805). The crystal structure of the GFP protein and of several point mutants has been solved (Ormo et al., 1996, Science 273, 1392; Yang et al., Nature Biotechnol. 14, 1246). The fluorophore, consisting of a tripeptide at residues 65-67, is buried inside a relatively rigid beta-can structure, where it is almost completely protected from solvent access. The GFP absorption bands and emission peak arise from an internal p-hydroxybenzylideneimidazolidinone chromophore, which is generated by cyclization and oxidation of the tripeptide sequence Ser-Tyr-Gly sequence at residues 65-67 (Cody et al., 1993, Biochemistry 32:1212-1218).

GFP fluorescence in prokaryotic and eukaryotic cells does not require exogenous substrates and cofactors. Accordingly, GFP is considered to have tremendous potential in methods to monitor gene expression, cell development, or as an in situ tag for fusion proteins (Heim et al., 1994, P.N.A.S. USA, 91, 12501-12504). Chalfie and Prasher, WO 95/07463 (Mar. 16, 1995), describe various uses of GFP, including a method of examining gene expression and protein localization in living cells. Methods are described wherein: 1) a DNA molecule is introduced into a cell, said DNA molecule having DNA sequence of a particular gene linked to DNA sequence encoding GFP such that the regulatory element of the gene will control expression of GFP; 2) the cell is cultured in conditions suitable for the expression of the fused protein; and 3) GFP expression is detected, thereby indicating the expression of the gene in the cell. Methods such as those described by Chalfie and Prasher are advantageous compared to previously reported methods which utilized β-galactosidase fusion proteins (Silhavy and Beckwith, 1985, Microbiol. Rev., 49, 398; Gould and Subramani, 1988, Anal. Biochem., 175, 5; Stewart and Williams, 1992, J. Gen. Microbiol., 138,1289) or luciferases, in that the need to fix cell preparations and/or add exogenous substrates and cofactors is eliminated. The sequence and structure of GFP quickly led to the discovery of mutants, or structurally similar proteins in other organisms, with different fluorescent properties.

GFP Fusion Proteins

The discovery that GFP can expressed and detected in living cells, without co-factors or substrates, rapidly led to the fusion of GFP to the C- or N-termini of other proteins. This revealed that GFP appended to the ends of other proteins produced functional, fluorescent proteins that can be imaged, and followed over time, in living cells. This provided temporal and spatial resolution of proteins in living cells that far exceeded any existing technology. The majority of GFP fusion proteins have been constructed by attaching, at the nucleic acid level, the two coding sequences such that the fusion protein places GFP at either the N- or C-terminus. However, several experiments have shown that an entire GFP can be inserted into the primary and secondary structure of the protein, and some of the resulting tribrid fusion proteins are both fluorescent and functional.

In two cases, internally inserted fluorescent protein has produced a new kind of biosensor. Fluorescent proteins inserted into a voltage gated potassium channel, or into a voltage gated sodium channel, can signal voltage driven rearrangements of the channel through changes in fluorescence (U.S. Pat. No. 6,660,844; Siegel, M S and Isacoff, E Y, Neuron: 4, 735-41 1997; Ataka et al., Biophys J (1):509-516 2002; Guerrero, G, Siegel, M S, Roska, B, Loots, E and Isacoff, E Y, Biophys J: 6, 3607-18 2002; each of which is herein incorporated by reference). The mechanism whereby the conformational changes of the channel are coupled to changes in fluorescence is poorly understood, but these experiments show that internally placed fluorescent proteins can generate new, genetically encoded biosensors of significant commercial value.

U.S. Pat. No. 6,180,343 relates to the use of fluorescent proteins, particularly green fluorescent protein (GFP), in fusion constructs with random and defined peptides and peptide libraries, to increase the cellular expression levels, decrease the cellular catabolism, increase the conformational stability relative to linear peptides, and to increase the steady state concentrations of the random peptides and random peptide library members expressed in cells for the purpose of detecting the presence of the peptides and screening random peptide libraries. The patent does not contemplate the use of antiparallel leucine zipper for reconstituting GFP nor the use of peptides that associate with each other to reconstitute GFP and to provide a detection signal.

Protein-Protein Interaction Assays

The yeast two-hybrid system for detecting protein-protein interactions in Saccharomyces cerevisiae (Fields and Song, 1989, Nature, 340:245-246; U.S. Pat. No. 5,283,173 by Fields and Song) is well known in the art. This assay utilizes the reconstitution of a transcriptional activator like GAL4 (Johnston, 1987, Microbiol. Rev., 51:458-476) through the interaction of two protein domains that have been fused to the two functional units of the transcriptional activator: the DNA-binding domain and the activation domain. This is possible due to the bipartite nature of certain transcription factors like GAL4. Being characterized as bipartite signifies that the DNA-binding and activation functions reside in separate domains and can function in trans (Keegan et al., 1986, Science 231:699-704). The reconstitution of the transcriptional activator is monitored by the activation of a reporter gene like the lacZ gene that is under the influence of a promoter that contains a binding site (Upstream Activating Sequence or UAS) for the DNA binding domain of the transcriptional activator. This method is most commonly used either to detect an interaction between two known proteins (Fields and Song, 1989, Nature, 340:245-246) or to identify interacting proteins from a population that would bind to a known protein (Durfee et al., 1993, Genes Dev., 7:555-569; Gyuris et al., 1993, Cell, 75:791-803; Harper et al, 1993, Cell, 75:805-816; Vojtek et al., 1993, Cell, 74:205-214). Like the ubiquitin system (U.S. Pat. No. 5,503,977, herein incorporated by reference), additional factors are required for detection of the protein-protein interaction. Additionally, in the yeast two-hybrid system, the protein interaction must occur in the nucleus of the yeast.

U.S. Pat. No. 5,362,625 discloses omega-acceptor and omega-donor polypeptides (comprising about two-thirds and one-third of the β-galactosidase molecule amino and carboxyl termini, respectively), prepared by recombinant DNA techniques, DNA synthesis, or chemical polypeptide synthesis techniques, which are capable of interacting to form an active enzyme complex having catalytic activity characteristic of β-galactosidase. The patent also describes the use of these polypeptides in enzyme complementation assays for qualitative and quantitative determination of a suspected analyte in a sample.

U.S. Patent Application Publication No. 2004/0235064 A1 describes the use of GFP fragments as a complementation assay. The description of this invention involves linking interacting protein domains to different fragments of the fluorescent protein. Protein interactions can be readily detected if the ends of these proteins bring the two fragments of the fluorescent protein close enough to one another. The novel aspect of this work is the observation that a single domain protein can be broken into fragments to produce a complementation assay. The proof of principle experiments were done by fusing small, interacting anti parallel leucine zippers to the two fragments of the Green Fluorescent Protein. Since then, analogous strategies have fused interacting transcription factors or G protein subunits to the ends of the GFP fragments (Hu C D, et al. Mol Cell (2002).9, 789-98; Hynes T R, et al. J Biol Chem (2004).279, 44101-12). The advantages of this approach over previous systems are several. First, the signal produced by the GFP can be readily detected in living cells. Second, the signal can be produced anywhere in the cell. Finally, the strategy can be used in many different cellular backgrounds, including human cells. This is important because many of the strategies, including the yeast 2 hybrid system for example, may be missing critical components of post translational processing that are necessary for the correct folding and trafficking of the fusion partners.

Complementation assays are a powerful way of studying and identifying protein interactions. Most of these assays depend upon bringing two different domains of a protein together to produce a detectable activity. While the GFP complementation system described in 2004/0235064 A1 has advantages over other known systems as discussed above, it will only work when the ends of the interacting proteins are physically close enough. The current invention extends this work by showing that, surprisingly, the fluorescent protein fragments can complement even when they are placed deep in the structure of two interacting proteins, for instance when the surfaces of the two interacting proteins are adjacent.

### SUMMARY OF THE INVENTION

The present invention involves the placement of complementing fragments of a reporter protein within the structure of two interacting proteins or two interacting protein surfaces or protein domains. When the reporter protein fragments are placed at interacting protein surfaces, the two fragments are brought close enough to one another to fold together to form a functional reporter protein. This approach does not rely upon attaching the complementing fragments to the ends of the interacting fragments. This has several advantages. First, there are protein interactions that will be blocked, due to steric hindrance, by the addition of reporter fragments to the ends of the interacting proteins. Second, protein interaction screens that depend upon placing the complimenting fragments at the ends of the fusion partners will only work when the ends of the interacting proteins are physically close enough to one another for the complementation to take place. Protein interactions in which the ends of the two proteins are too far apart will be lost. Third, appending the reporter fragments to the interacting fragments often involves fragmenting or truncating the interacting proteins. The present invention works with full length proteins, where at least one complementing fragment is fused at an internal position within the interacting protein.

Thus, the present invention concerns methods of detecting interaction of at least a first and second protein or protein domain by complementation of at least two fragments of a reporter protein, wherein at least two fragments of said reporter protein are genetically fused to the first and second proteins or protein domains, respectively, and at least one complementing fragment is fused at an internal position within one of the first or second interacting proteins or protein domains. The present invention also encompasses methods wherein each of the complementing fragments is fused at an internal position within one of the interacting proteins or protein domains. While there are many ways to construct the interacting fusion proteins of the invention, as described herein, a convenient means of construction is to use a transposon to randomly insert each complimenting fragment into each member of a pair of interacting proteins. This enables one to quickly and easily generate a library of fusions containing the complimenting fragments fused at different locations, and screen this library for interacting members by observing those that bring the complimenting fragments of the reporter together to form a functional reporter protein.

In this regard, the present invention also encompasses a transposable complementation system for detecting protein interactions comprising at least one transposon encoding at least one complementing fragment of a reporter protein, and kits comprising the same. The transposable complementation system of the invention may include at least two transposons, wherein each transposon encodes at least one complementing fragment of a reporter protein. Alternatively, the transposable complementation system of the invention may include a single transposon encoding at least a first and a second complementing fragment of a reporter protein. In the single transposon systems of the invention, in-frame fusions to each complementing fragment may be generated by removing other regions of the transposon after transposition into the target gene, for instance by restriction digestion.

To the inventors' knowledge the present invention is the first to provide a complementation assay for studying protein interactions where the complementing fragments of the reporter protein are genetically fused at internal positions within interacting proteins or interacting protein domains of one or more proteins. Accordingly, the present invention also encompasses an isolated nucleic acid which encodes a fusion protein comprising the sequence of a first member of a binding pair fused to at least a first complementing fragment of a reporter protein and methods of making the same, wherein the complementing fragment does not produce detectable reporter protein activity in the absence of at least one other complementing fragment that is not present in said fusion protein or in the same protein domain as said first complementing fragment, and wherein the complementing fragment is fused to the binding member at an internal site of the binding member. A single nucleic acid may encode two or more separate complementing fragments that are located in different protein domains, for instance for assaying protein folding or changes in protein conformation.

The present invention also encompasses a fusion protein encoded by such a nucleic acid, as well as a mixture comprising the fusion protein and at least a second fusion protein comprising the sequence of a second member of the binding pair fused to a second complementing fragment of the reporter protein, wherein the second complementing fragment does not produce detectable reporter protein activity in the absence of the first complementing fragment and the first complementing fragment is not present in said second fusion protein, and wherein the first and second complementing fragments do not have affinity for one another in the absence of said binding pair. In the mixture of the invention, the second complementing fragment may also be fused to the second binding member at an internal site of the second binding member. Only by binding of the first binding member to the second binding member is detectable reporter protein activity produced, via complementation of said first and second complementing fragments. Assays could also be designed using more than two complementing fragments, where none of the complementing fragments has affinity for another complimenting fragment, and wherein all complementing fragments are brought together to produce functional reporter protein activity by interacting members of a binding complex. The mixtures of the invention may also be used as biosensors for detecting a change in concentration of an analyte having specificity for the binding pair.

The present invention is useful for detecting or measuring the binding of two or more interacting proteins, for instance by measuring functional reporter protein activity following complementation of the complementing fragments fused to each binding member. The methods of the invention are also useful for measuring or detecting protein folding, for instance, where the two or more protein domains are located in a single protein. The present invention also includes methods for measuring dimerization or aggregation of protein subunits, for instance, where the complementing fragments are located in two or more subunits of a dimeric or multimeric protein complex, including receptor complexes. The present invention also includes methods for detecting or measuring the interaction of a ligand with its receptor, for instance where the complementing fragments are located in interacting ligand and receptor proteins, respectively. The methods of the present invention may also be used to detect protein interactions within a cell, for instance where the complementing fragments are located in different proteins that participate in a common signal transduction pathway. The constructs of the present invention may also be used in methods of identifying compounds that modulate binding of a first protein to a second protein, for instance by observing changes in reporter protein activity via complementation of fragments in the first and second proteins in response to exposure to different compounds. Such assays may be readily performed at the high throughput level.

### BRIEF DESCRIPTION OF THE DRAWINGS

FIG. 1 is a diagram showing the mosaic ends of the hyperactive Tn5 transposon (SEQ ID Nos. 1 and 2, respectively).

FIG. 2 is a diagram of one transposable complementation system according to the present invention.

FIG. 3 is a diagram showing digestion of an in-frame insertion to generate fusions to either the first or second fragment of a reporter protein.

FIG. 4 is a diagram of the location of 29 unique transposon insertions obtained in the Shaker ion channel.

FIG. 5 is a diagram showing the complementation pattern of one Shaker subunit pair that produced fluorescence.

FIG. 6 is a diagram showing that a single insertion of one portion of the fluorescent protein reporter is able to complement with other subunits carrying the second portion in different locations.

FIG. 7 is a diagram showing that complementation patterns of complementing Shaker subunits are not always reciprocal.

FIG. 8 is a diagram of the total pattern of complementation observed for insertions in the Shaker ion channel.

FIG. 9 is a diagram pointing out noise in the system, where two pairs of complementing fragments at positions normally expressed on the extra and intracellular sides of the channel led to low level fluorescence when retained in the endoplasmic reticulum.

FIG. 10 is a photograph comparing fluorescence from a Shaker complementing pair as compared to a membrane targeted red fluorescence protein.

FIG. 11 is a graph showing the response of HIEK 293 cells expressing a pair of complementing subunits in response to depolarization with extracellular potassium.

FIG. 12 is a graph of voltage versus gating measurements showing that some pairs of complementing subunits exhibit normal channel activity.

### DETAILED DESCRIPTION OF THE INVENTION

The invention places complementing halves of the fluorescent protein deep within the structure of the two interacting proteins rather than attaching them to the ends of the interacting proteins. Our data reveal that if the fluorescent protein fragments are inserted into adjacent surfaces of two different proteins they can complement one another to form a functional fluorescent protein. This approach can be used with large proteins that exist anywhere inside or outside of the cell, and it does not require that the C- or N-termini of the interacting proteins are adjacent to one another. One potential market for this invention is already defined by those who are using other complementation strategies, and the invention will make it possible to detect protein interactions that are currently being missed with the existing technologies.

The invention encompasses any method of detecting protein interaction involving complementation of internally fused reporter protein fragments. For example, the methods of the invention include a method of detecting interaction of at least a first and second protein or protein domain by complementation of at least two fragments of a reporter protein, wherein the at least two fragments of the reporter protein are genetically fused to the first and second proteins or protein domains, respectively, and at least one complementing fragment is fused at an internal position within one of the first or second interacting proteins or protein domains. Also included are methods wherein each of the at least two complementing fragments is fused at an internal position within one of the two or more interacting proteins.

In the methods of the present invention, the complementing fragments of the reporter protein have no affinity for one another in the absence of the interacting proteins or protein domains. Accordingly, “complementation” refers to the functional reassembly of separate fragments into an active enzyme. Fragments are considered to be “complementing fragments” when they are not part of a single protein, when they have no natural affinity for one another, but they are able to assemble and form a functional protein when brought into close contact.

According to the invention, “interaction of at least a first and second protein” means that at least two proteins come into close enough contact for a sufficient time and with a sufficient stability so as to facilitate complementation of two or more fragments of a reporter protein, thereby resulting in detectable, measurable reporter protein function or activity. Interaction of at least a first and second protein “domain” means that at least two protein domains come into close enough contact for a sufficient time and with a sufficient stability so as to facilitate complementation of two or more fragments of a reporter protein, thereby resulting in detectable, measurable reporter protein function or activity, wherein the domains may be present in a single protein or more than one protein. According to the invention, a protein “domain” is an element of overall structure that is self-stabilizing and often folds independently of the rest of the protein chain. The complementing fragments used in the methods of the invention may also be located in interacting regions of a single protein domain. Two proteins or protein domains that interact for a sufficient time and with a sufficient stability so as to facilitate complementation of two or more fragments of a reporter protein may be referred to as “interacting proteins” or protein domains or “binding members.” Putative interacting proteins may also be employed in the methods of the present invention, for instance for testing the interaction of proteins that are only suspected of interacting, or for testing libraries of proteins for those that interact with a given receptor, ligand, analyte or binding member.

In the methods of the present invention, the fragments of the reporter protein are genetically fused to the first and second proteins or protein domains, respectively. “Genetically fused” in the context of the present invention means that a nucleic acid encoding a reporter protein fragment is fused “in-frame” with a nucleic acid encoding an interacting protein such that there are no stop codons between the sequence for the interacting protein and the sequence for the reporter protein fragment, and the two are expressed as a single “fusion protein.” In the methods of the present invention, at least one complementing fragment is fused at an internal position within one of the interacting proteins, meaning that the reporter protein fragment is not fused to either the amino or carboxyl-terminus but within the open reading frame of the interacting protein such that there are amino acids from the interacting protein on both ends of the reporter protein fragment. An “internal position” refers to any position within the coding region of the interacting protein, at a distance of one or more amino acids from either end of the interacting protein. For instance, a complementing fragment may be fused at an internal site that is at least about 5, at least about 10, at least about 20, at least about 30, at least about 40, at least about 50, at least about 75, at least about 100, at least about 150, at least about 250, or at least about 500 amino acids or more from either the amino or carboxyl terminus of the interacting protein or binding member.

Any conceivable method may be employed to engineer the interacting fusion proteins of the present invention. For instance, the skilled artisan may readily design and chemically synthesize linker nucleic acids (single stranded) and/or adapter nucleic acids (double stranded) for facilitating insertion of DNA encoding reporter protein fragments into the open reading frame of a gene or cDNA encoding a selected interacting protein by incubating the reporter fragment DNA, cleaved DNA encoding the interacting protein and any suitable linker or adapter with a ligase enzyme using methodology known in the art. The nucleic acids encoding the reporter protein fragments may be inserted at any known restriction site with the knowledge of the gene sequence of the interacting protein in hand. Linkers and adapters may be easily designed to facilitate insertion of reporter protein encoding fragments into any restriction site, with the length and codon content of the linker or adaptor being designed to maintain the open reading frame, structure and/or function of the resulting fusion protein. Alternatively, the DNA encoding the interacting protein may be physically sheared, for instance using hydrodynamic forces, sonication, shaking or vortexing, and the reporter protein encoding fragment inserted into the sheared DNA using blunt-end ligation and/or gap-fill reactions.

Protein linkers may also be engineered into the fusion proteins and employed to facilitate separate folding of the complementing fragment within the interacting protein. Such linkers may include from at least about one, at least about two, at least about three, at least about four, at least about five, at least about seven, at least about ten, at least about fifteen, at least about twenty up to about fifty amino acids or more.

A particularly convenient way to generate a functional complementing pair of fusion proteins, especially when one does not know which locations in a given binding member will be conducive for facilitating complementation, is to use transposon insertion. By using a transposon encoding the complementing fragment, the fragment nucleic acid may be introduced at random locations within the gene or cDNA for a given binding member, or within the members of a library of binding member nucleic acids, and the resulting fusions expressed and screened for those that facilitate complementation by detecting function reporter protein activity. A selectable marker may be used to select for nucleic acids receiving a transposon insertion, and the selectable marker may be removed following transposition to recreate the fused reading frame using restriction digestion or site-specific recombination, for instance using a recombinase such as Cre recombinase or FLP recombinase. To the inventors' knowledge, complementing fusion proteins have never before been generated using transposons, as it is not intuitive that two interacting proteins containing internally fused complementing fragments of a reporter protein could facilitate reassembly of a functional reporter protein.

Thus, the present invention also includes a transposable complementation system for detecting protein interactions comprising at least one transposon encoding at least one complementing fragment of a reporter protein. The transposable complementation system of the present invention may comprise at least two transposons, wherein each transposon encodes at least one complementing fragment of a reporter protein. Alternatively, the transposable complementation system of the invention may comprise a single transposon encoding at least a first and a second complementing fragment of a reporter protein, wherein separate fusions to each complementing fragment may be generated from a single transposon insertion following removal of intervening transposon DNA by restriction digestion or site-specific recombination.

In one embodiment of the invention, among others, the transposable complementation system comprises one or more selectable marker genes. In single transposon systems, the one or more selectable marker genes may be located anywhere on the transposon, for instance between the nucleic acid sequences encoding the two or more complementing fragments. The nucleic acid sequences encoding the complementing fragments may also be contained in a longer open reading frame encoding a functional reporter protein, wherein expression of the functional reporter protein serves as one marker of transposon insertions that generate in-frame fusions. Subsequent cleavage of the transposon DNA could then be used to generate fusions to the one or more complementing fragments originally contained in the longer reporter protein open reading frame to generate a fusion protein containing only the complementing fragment for use in complementation assays.

In one embodiment of the present invention, the transposable complementation system employs one or more transposons comprising a sequence of elements according to the formula:

5′ R1-F1-R2-S—R1-F2-R2 3′

wherein R1 and R2 are first and second, preferably unique, restriction sites, respectively; F1 and F2 are nucleic acid sequences encoding complementing fragments of a reporter protein; and S is at least one selectable marker gene. In the context of the present invention, “unique” means that the two cleavage sites for R1 and R2, respectively, are the only such cleavage sites for those enzymes in the transposon. It is also possible to use restriction enzymes that cut more frequently, for instance by limiting the restriction reaction by time or amount of enzyme and screening for those cleavage reactions that generate complementing fragments. Restriction digestion following transposition using a restriction enzyme that cleaves at R1, and subsequent ligation to remove the internal transposon fragment, will generate an in-frame fusion of the nucleic acid encoding the selected interacting protein with the nucleic acid encoding the second complementing fragment, F2. Restriction digestion following transposition using a restriction enzyme that cleaves at R2, and subsequent ligation to remove the internal transposon fragment, will generate an in-frame fusion of the nucleic acid encoding the selected interacting protein with the nucleic acid encoding the first complementing fragment, F1. In this manner, a single transposon insertion may be used to generate fusions of an interacting protein (IP) to each complementing fragment at the same insertion point, to yield fusion proteins of the following formulas:

5′-IP1-R1-F1-IP2-3′ and 5′-IP1-F2-R2-IP2-3′

wherein IP1 and IP2 are the regions of the nucleic acid encoding the interacting protein on either side of the nucleic acid encoding the complementing fragment, F1 or F2, respectively.

In another embodiment, the first complementing fragment F1 is contained in an open reading frame encoding a functional reporter protein such that in-frame transposition of the transposon into an open reading frame results in expression of a functional reporter protein. Such transposable complementation systems employ one or more transposons comprising a sequence of elements according to the formula:

5′—R1-F1-R2-F2-R1-F2-R2-3′

wherein R1 and R2 are first and second, preferably unique, restriction sites, respectively; F1 and F2 are nucleic acid sequences encoding complementing fragments of a reporter protein with the left-most (5′) F2 nucleic acid in-frame with the nucleic acid encoding F1; and S is at least one selectable marker gene. In-frame transposition events into an open reading frame will generate detectable reporter protein activity prior to assaying for complementation via fusion to the elements 5′—R1-F1-R2-F2-3′. Restriction digestion following transposition using a restriction enzyme that cleaves at R1, and subsequent ligation to remove the internal transposon fragment, will generate an in-frame fusion of the nucleic acid encoding the selected interacting protein with the nucleic acid encoding the right-most (3′) second complementing fragment, F2. Restriction digestion following transposition using a restriction enzyme that cleaves at R2, and subsequent ligation to remove the internal transposon fragment, will generate an in-frame fusion of the nucleic acid encoding the selected interacting protein with the nucleic acid encoding the first complementing fragment, F1. In this manner, a single transposon insertion may be used to generate fusions of an interacting protein (IP) to each complementing fragment at the same insertion point, to yield fusion proteins of the following formulas:

5′-IP1-R1-F1-IP2-3′ and 5′-IP1-F2-R2-IP2-3′

wherein IP1 and IP2 are the regions of the interacting protein on either side of the complementing fragment, F1 or F2, respectively.

Using the complementation systems of the present invention, protein interactions may be detected in or on any type of cell, including but not limited to prokaryotic cells, including fungi, yeast and bacteria such as Escherichia coli, and eukaryotic cells, including plant cells and animal cells, including but not limited to human, murine, primate, rat, rabbit, guinea pig, bovine, ovine, and equine to name a few. One of ordinary skill in the art is well aware of how to clone nucleic acids encoding a particular interacting fusion proteins into one or more appropriate expression vectors and transfect the one or more vectors into a particular cell of interest to express the interacting fusion proteins. Protein interactions may also be detected in vivo in any kind of plant or animal, in both vertebrate and invertebrate organisms, for instance in transgenic organisms engineered to express the interacting fusion proteins in the same cell type using developmentally regulated or tissue-specific promoters, or by delivering one or more vectors encoding the interacting fusion proteins to a target tissue by direct injection or by use of a delivery vehicle such as a liposome or a nanoparticle. Accordingly, the reporter protein, selectable marker(s) and/or type of transposon employed may be selected and modified depending on the host cell or animal in which complementation is to be screened.

Any suitable reporter protein may be employed in the methods of the invention so long as the protein may be split into two or more fragments that have no natural affinity for one another, but that reassemble when in close proximity to form a function reporter protein. Exemplary reporter proteins that may be used in the complementation systems and methods of the invention include but are not limited to reporter proteins selected from the group consisting of GFP (green fluorescent protein) (Ghosh et al. 2000 J. Am. Chem. Soc 122:5658-59) and derivatives thereof, including BFP (blue fluorescent protein), CFP (cyan fluorescent protein) and YFP (yellow fluorescent protein) (Hu, and Kerppola 2003 Nat. Biotechnol. 21: 539-545), and enhanced variants thereof, i.e., YFP-Venus (Nagai et al. 2002 Nat. Biotechnol. 20: 87-90), dsRed (Jach et al., 2006, Nat. Methods 3: 597-600), Citrine, and variants thereof. As used herein, the term “variant” is intended to refer to polypeptides with at least about 30%, at least about 40%, at least about 50%, at least about 75% identity, at least about 85%, at least about 90%, at least about 95% or greater identity to native fluorescent molecules. Many such variants are known in the art, or can be readily prepared by random or directed mutagenesis of native fluorescent molecules (see, for example, Fradkov et al., FEBS Lett. 479:127-130 (2000)). Other types of reporter proteins may also be used, including dihydrofolate reductase (DHFR), beta-lactamase, luciferase and β-galactosidase.

In one embodiment of the invention, among others, the reporter protein is GFP or any of its derivatives, for instance YFP Venus, and the complementing fragments are generated by splitting GFP or its derivative between amino acids 158 and 159 of the full length protein. Other locations to split GFP and its variants and generate complementing fragments are certainly possible and are within the scope of the present invention. See, for instance, U.S. Pat. No. 6,780,599 and US 2004/0235064, which describe complementation using GFP which is dissected at surface loop between amino acid residues 157 and 158, and are herein incorporated by reference in their entireties. Based on a variety of studies that have circularly permuted GFP, which have split GFP, or which have inserted peptides into GFP, there is good evidence that GFP can be split in several different places to produce a complementation system. Similarly, other fluorescent proteins that are quite analogous in structure can probably be split at structurally similar places. Such locations include, but are not limited to between amino acids 38-39 (Hu C D, et al. (2002) Visualization of interactions among bZIP and Rel family proteins in living cells using bimolecular fluorescence complementation. Mol Cell 9:789-98); between amino acids 101-102 (Hu et al. (2002)); between amino acids 128-129 (Ozawa, T., Nogami, S., Sato, M., Ohya, Y. and Umezawa, Y. (2000). A fluorescent indicator for detecting protein-protein interactions in vivo based on protein splicing. Anal. Chem. 72: 5151-5157; Ozawa, T., Sako, Y., Sato, M., Kitamura, T. and Umezawa, Y. (2003). A genetic approach to identifying mitochondrial proteins. Nat. Biotechnol. 21: 287-293; Ozawa, T., Takeuchi, T. M., Kaihara, A., Sato, M. and Umezawa, Y. (2001). Protein splicing-based reconstitution of split green fluorescent protein for monitoring protein-protein interactions in bacteria: improved sensitivity and reduced screening time. Anal. Chem. 73: 5866-5874); between amino Acids 144-145 (Hu et al. (2002); Ozawa et al. (2000); Ozawa et al. (2003) Ozawa et al. (2001)); anywhere in the 9 amino acid loop constituting amino acids 153-161, which includes at least the following split sites: amino acids 154-155 (Hu et al. (2002)); and amino acids 157-158 (Magliery T J, et al. (2005) Detecting protein-protein interactions with a green fluorescent protein fragment reassembly trap: scope and mechanism. J Am Chem Soc 127:146-57; Ghosh I, et al. (2000) Antiparallel Leucine Zipper-Directed Protein Reassembly: Application to the Green Fluorescent Protein. J Am Chem Soc 122:5658-9; Hu et al. (2002) Visualization of interactions among bZIP and Rel family proteins in living cells using bimolecular fluorescence complementation. Mol Cell 9:789-98); amino acids 192-193 (Hu et al. (2002) Visualization of interactions among bZIP and Rel family proteins in living cells using bimolecular fluorescence complementation. Mol Cell 9:789-98); amino acids 214-214 (Cabantous S, Waldo G S. (2006) In vivo and in vitro protein solubility assays using split GFP. Nat Methods 3:845-54; Cabantous S, et al. (2005) Protein tagging and detection with engineered self-assembling fragments of green fluorescent protein. Nat Biotechnol 23:102-7; Cabantous S, et al. (2005) Recent advances in GFP folding reporter and split-GFP solubility reporter technologies. Application to improving the folding and solubility of recalcitrant proteins from Mycobacterium tuberculosis. J Struct Funct Genomics 6:113-9); amino acids 224-225 (Ozawa et al. (2000); Ozawa et al. (2003); Ozawa et al. (2001)). Red fluorescent protein has also been split at the loop most used for GFP: amino acids 154-155 and amino acids 168-169 (Jach G, et al. (2006) An improved mRFP1 adds red to bimolecular fluorescence complementation. Nat Methods 3:597-600).

Complementation using fragments of β-galactosidase has also been described. See U.S. Pat. No. 6,342,345, which is herein incorporated by reference in its entirety. Complementation using fragments of DHFR, beta-lactamase and luciferase has also been described. See U.S. Pat. No. 7,062,219, which is herein incorporated by reference in its entirety. However, none of these references describes or contemplates the systems or methods of the present invention, where at least one or all complementing fragments are internally fused within the interacting proteins.

The transposon employed in the system of the invention may also vary, and may be selected and modified depending on the host cell or animal in which complementation is to be screened. Transposition reactions may be performed in the host cell, for instance by introducing the transposon on a vector with the transposase supplied in trans either on the chromosome or the vector, with selectable markers and other transposon DNA removable following transposition by site-specific recombination as discussed above. Alternatively, transposition reactions may be performed in vitro into an isolated nucleic acid target or library of target nucleic acids, by exposing the target nucleic acid(s) to the transposon and isolated transposase enzyme. Nucleic acids receiving transposon insertions may then be selected by transforming the nucleic acids from the reaction into a suitable host cell and selecting cells expressing the selectable marker on the transposon.

Depending on the cell or organism in which complementation is assayed, any type of transposon may be used in the transposable complementation systems of the present invention, including but not limited to bacterial transposons and eukaryotic transposons, including insect, animal and plant-derived transposons. If one is performing transposition reactions in a host cell, a transposon/transposase pair that is active in that particular cell type may be chosen. Alternatively, if one is introducing into the cell a gene encoding a fusion protein that already contains transposon DNA in a desired position, one may want to chose a transposon that is foreign to the host cell to avoid unwanted recombination events or subsequent undesirable transposition

There are a wide variety of transposons that are useful for the methods of the present invention. Insect or animal-derived transposons useful in the systems of the invention include Mariner-type transposons, Sleeping Beauty transposons, piggyBac transposons, Tigger transposons, pogo transposons and Alu elements, among others. Plant-derived transposons include but are not limited to activator transposons (Ac), mutator transposons (Mu), mutator-like elements (Mules), Suppressor mutator (Spm) transposons, Enhancer/Suppressor (En/Spm) transposons, Tam1, Tam2, and Tam3 transposons, to name a few. Bacterial transposons useful for the systems of the present invention include any known bacterial transposon. These include, but are not limited to, Tn10 (Huisman O, et al. (1987) A Tn10-lacZ-kanR-URA3 gene fusion transposon for insertion mutagenesis and fusion analysis of yeast and bacterial genes. Genetics 116:191-9), mTn (Ross-Macdonald P, et al. (1997) A multipurpose transposon system for analyzing protein production, localization, and function in Saccharomyces cerevisiae. Proc. Natl. Acad. Sci. USA 94:190-5.), as well as Tn3 (Hoekstra M F, et al. (1991) A Tn3 derivative that can be used to make short in-frame insertions within genes. Proc. Natl. Acad. Sci. USA 88:5457-61), Tn5 and Tn4430 (reviewed in: Manoil C, Traxler B. (2000) Insertion of in-frame sequence tags into proteins using transposons. Methods 20:55-61). A suitable transposon may be chosen from any of the publicly available databases. For instance, ACLAME (A CLAssification of genetic Mobile Elements) is a database dedicated to the collection and classification of mobile genetic elements (MGEs) from various sources, including all known phage genomes, plasmids and transposons. Tn5 derivatives are particularly suitable for in vitro transposition reactions as Tn5 transposase is commercially available in isolated form (EZ-Tn5™, Epicentre Biotechnologies; see, e.g., U.S. Pat. Nos. 5,925,545; 5,948,622; 5,965,443; 6,437,109; 6,159,736; and 6,294,385, which are each herein incorporated by reference in their entireties). The most useful transposons will: 1) show little preference for insertion site, in order to maximize the efficiency of the screen for appropriate insertion sites; 2) show a high frequency of insertion into other sequences of DNA; 3) contain at least one reading frame across the DNA sequences that flank the transposon sequence and which are incorporated in the DNA that is ultimately inserted into the target. This is critical for producing a fusion protein in which the sequence encoding the target protein, and the one encoding the reporter fragment, are joined to produce one continuous reading frame that encodes a single fusion protein.

Selectable marker genes may also vary depending on the host cell to be used. For instance, suitable selectable marker genes include but are not limited to kanamycin, tetracycline, gentamycin, phleomycin, erythromycin, clindamycin, neomycin, chloramphenicol, zeocin, as well as any genetic elements that can be used for selection such as supF (Merkulov G V, Boeke J D. (1998) Libraries of green fluorescent protein fusions generated by transposition in vitro. Gene 222:213-2.) or URA3. It is also possible to use transposons that do not contain selectable marker genes, for instance where the transposon has a high rate of transposition and/or where the initial transposition event generates an in-frame functional fusion protein whose activity may be screened directly without selection. It is also possible to isolate nucleic acids receiving transposon insertions by including in the transposon a nucleic acid tag that may be used to isolate transposon-containing DNA by hybridization to a solid support, or by sandwich hybridization using methods known in the art. See, for instance, U.S. Pat. No. 6,221,581, which is herein incorporated by reference in its entirety.

As discussed above, restriction digestion is one means to remove extraneous transposon DNA following a transposition event. The skilled artisan will understand that any known restriction enzyme may be used so long as it cleaves the transposon in the appropriate places to generate the complementing fragments, and does not cleave the target nucleic acid encoding the interacting protein. Transposons and reporter protein genes may be genetically engineered using site-directed mutagenesis and techniques known in the art to contain one or more restriction sites suitable for generating the complementing fragments. The skilled artisan also knows how to design new restriction sites in a protein coding sequence that maintain the open reading frame and the structure/function of the encoded protein or protein fragments.

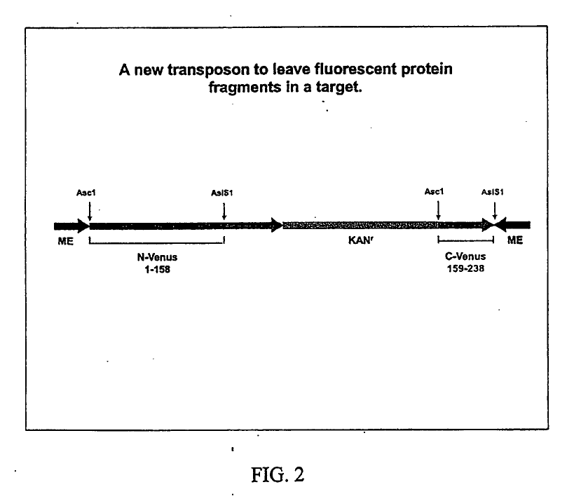

In one embodiment of the invention, among others, the transposon system is a Tn5 or other transposon system encoding complementing fragments of GFP or a derivative thereof, wherein the GFP fragments have been genetically engineered to contain unique restriction sites R1 and R2 at the boundaries flanking the sequences encoding the complementing fragments. In one embodiment, the transposon is a Tn5 derivative that has been genetically engineered to insert restriction sites R1 and R2, where R1 is an AscI restriction site and R2 is an AsiSI restriction site (see FIG. 2). These two restriction sites may be used to generate complementing fragments of GFP or any of its derivatives, including YFP Venus, wherein F1 consists of amino acids 1 to 158 of GFP and F2 consists of amino acids 159 to 238 of GFP. Of course, other restriction sites in GFP, or other sites engineered into GFP, may be useful depending on the boundaries of the desired complementing fragments. Any known restriction sites and enzymes may be employed depending on the sequences of the target nucleic acid, the transposon and the reporter gene used. Preferably, these sites are unique as described above, however, it is also possible to use restriction enzymes that cut more frequently, for instance by limiting the restriction reaction by time or amount of enzyme and screening for those cleavage reactions that generate complementing fragments.

The present invention also includes a kit comprising a transposable complementation system of the invention. Such kits may also comprise at least one transposase enzyme specific for the transposon or transposons provided in the kit, or a gene encoding a transposase that can catalyze transposition of the transposon or transposons. In one embodiment, among others, a kit of the invention comprises, in addition to the one or more transposons, restriction enzymes R1 and R2. The kits of the invention may further comprise instructions for isolating one or more transposon insertions in a target nucleic acid or group of target nucleic acids, and instructions for screening for complementation and reporter protein activity.

Thus, the methods of the invention include the use of the transposable complementation systems of the invention for detecting protein interactions. In one embodiment, among others, the invention includes a method of detecting interaction of two or more protein domains using a transposable complementation system of the invention, comprising:

(a) inserting randomly into one or more target nucleic acids one or more transposons encoding at least a first complementing fragment of a reporter protein;

(b) expressing said one or more target nucleic acids receiving said transposon insertions to produce one or more fusion proteins containing said first complementing fragment of said reporter protein;

(c) exposing said one or more fusion proteins containing said first complementing fragment of said reporter protein to one or more fusion proteins containing one or more other complementing fragments of said reporter protein; and

(d) detecting reporter protein activity; wherein said first complementing fragment complements said one or more other complementing fragments to form a functional reporter protein, wherein said first complementing fragment and said one or more other complementing fragments have no natural affinity for one another in the absence of interaction of said two or more protein domains, and wherein detectable activity of said reporter protein indicates interaction of said two or more protein domains. As indicated above, the two or more protein domains may be located in a single protein. Alternatively, the two or more protein domains may be located in two or more subunits of a single protein complex, such as dimeric or multimeric receptor complexes, including but not limited to ion channels and G protein coupled receptors. The two or more protein domains may also be located in two or more different proteins, for instance putative ligand and receptor proteins, respectively, or two or more different proteins that participate in a common signal transduction pathway.

It will also be possible to use different reporters of the present invention to simultaneously analyze multiple protein interactions where the different reporter proteins employed provide fluorescence of different colors. For example, one may simultaneously detect or measure the interaction of one protein pair using CFP complementing fragments and the interaction of a second protein pair using YFP complementing fragments. Any complementing pairs that produce different fluorescent proteins, which can be distinguished by excitation or emission properties, a combination thereof, or fluorescence lifetimes, can be used to simultaneously detect many different kinds of protein interactions. Widely available filter sets for microscopes currently make it possible to efficiently and inexpensively detect and distinguish between three different fluorescent proteins that can be used in the invention, CFP, YFP and RFP. Spectral imaging can be used to distinguish between at least 10 different fluorescent proteins, making it possible to detect at least different protein interactions simultaneously.

While there may be any number of complementing fragments that reassemble to form the functional reporter protein, for instance, two, three, four or more, the simplest systems will employ two complementing fragments. Accordingly, in some embodiments of the invention, in step (c), the one or more fusion proteins containing said first complementing fragment of said reporter protein are exposed to one or more fusion proteins containing a second complementing fragment of said reporter protein; wherein the first complementing fragment complements said second complementing fragment to form a functional reporter protein, and wherein detectable activity of said reporter protein indicates interaction of two or more protein domains. In one embodiment of the invention, the first and second complementing fragments are fragments of GFP or a derivative thereof, however, any complementing fragments of any suitable reporter protein may be used, such as any of those described above. In one embodiment, the first fragment consists essentially of amino acids 1 to 158 of YFP Venus and the second fragment consists essentially of amino acids 159 to 238 of YFP Venus. However, other fragments of GFP or YFP or any other GFP derivative may be used, as described above.

The methods of the present invention need not employ transposons to generate all interacting fusion proteins. Any of the above described methods for generating fusions may be combined with the use of transposon-based systems for detecting protein interactions. For instance, in one embodiment, the one or more fusion proteins containing the one or more other complementing fragments of said reporter protein or the second complementing fragment (step c of the above method) are generated by restriction digestion and ligation of the complementing fragment into the gene for the second interacting protein. This would be useful in instances where a panel of fusions containing random insertions of the first complementing fragment is screened for interaction with a second complementing fragment that is placed at a set location within the second interacting protein. Alternatively, the transposon systems of the invention may be used to generate both fusions, and the one or more fusion proteins containing the second complementing fragment of the reporter protein may also be expressed from one or more nucleic acids comprising one or more transposons encoding the second complementing fragment. This would be useful, for instance, where a panel of fusions containing random insertions of the first complementing fragment is screened for interaction with a panel of fusions containing random insertions of the second complementing fragment.

The methods of the invention may also include the additional steps for selecting and removing selectable marker genes. For instance, in step (a) above, target nucleic acids receiving transposon insertions may be isolated by selecting for expression of one or more selectable marker genes on the transposon. The selectable marker gene may be any suitable selectable marker gene, for instance an antibiotic resistance gene. In some embodiments, for instance those employing Tn5 transposon derivatives, the selectable marker encodes kanamycin resistance. In some embodiments where selection of a selectable marker is employed, after step (a) but before step (b), the selectable marker genes on the transposon insertions are removed from each nucleic acid such that expression in step (b) generates fusion proteins that comprise said first complementing fragment at an internal position in each expressed protein. Selectable marker genes and other intervening DNA may be removed by restriction digestion or site-specific recombination as described above. Of course, in embodiments where the second or other fusion protein comprising the second or other complementing fragment(s) is prepared by transposon insertion additional steps for selecting and removing selectable marker genes. Further, the methods of the invention may also employ any of the transposable complementation systems of the invention as described above wherein the one or more transposons encoding at least a first complementing fragment of a reporter protein and said one or more transposons encoding said second complementing fragment are the same transposon, and the transposon is cleaved differently following insertion to generate fusion proteins that comprise only said first complementing fragment or only said second complementing fragment, respectively.

To the inventors' knowledge the present invention is the first to provide a complementation assay for studying protein interactions where the complementing fragments of the reporter protein are genetically fused at internal positions within interacting proteins or interacting protein domains of one or more proteins. Accordingly, the present invention also encompasses an isolated nucleic acid which encodes a fusion protein comprising the sequence of a first member of a binding pair fused to at least a first complementing fragment of a reporter protein and methods of making the same, wherein the complementing fragment does not produce detectable reporter protein activity in the absence of at least one other complementing fragment that is not present in said fusion protein or in the same protein domain as said first complementing fragment, and wherein the complementing fragment is fused to the binding member at an internal site of the binding member. In the nucleic acid of the invention, the complementing fragment encoded by the nucleic acid is fused at an internal site that is at least one amino acid away from either terminus, or at least about 2, at least about 3, at least about 5, at least about 10, at least about 20, at least about 30, at least about 40, at least about 50, at least about 75, at least about 100, at least about 150, at least about 250, or at least about 500 amino acids or more from either the amino or carboxyl terminus of said binding member. A single nucleic acid may encode two or more separate complementing fragments that are located in different protein domains, for instance for assaying protein folding or changes in protein conformation, or located within the same domain, for instance for assaying folding and function of a particular domain within a protein.

As discussed above, there are many different ways of constructing the nucleic acids of the invention. Thus, the present invention encompasses any method of making a nucleic acid encoding an interacting protein comprising at least one internally fused complementing fragment of a reporter protein, comprising inserting into said nucleic acid a coding sequence for said complementing fragment such that said coding sequence is in frame with the sequence encoding said interacting protein. Again, “internally fused” in the context of the present invention means that the sequence of the complementing fragment is fused in frame at both the amino and carboxyl termini with coding sequence of the interacting protein.

Any conceivable method may be used to make the nucleic acids of the invention, as described above, including restriction digestion and ligation, with or without the use of linkers or adaptors. Also, the DNA encoding the interacting protein may be physically sheared to create insertion points for ligating the DNA encoding the complementing fragment. Insertion of the DNA encoding the complementing fragment may be site-directed to a specific location, or the DNA may be inserted at any random location within the coding sequence of the interacting protein.

In one embodiment, among others, the invention encompasses a method of making a nucleic acid encoding an interacting protein comprising at least one internally fused complementing fragment of a reporter protein, comprising:
1) exposing nucleic acids encoding said interacting protein to a transposon encoding said complementing fragment and a transposase such that transposition of said transposon into said nucleic acids occurs; and
2) screening said nucleic acids for those containing an in-frame transposon insertion.
   Transposition reactions may be performed in vitro, for instance with purified transposase, or in a cell expressing the transposase. Nucleic acids containing an in-frame insertion may be screened by isolating and transfecting said nucleic acids into an appropriate host cell to select for nucleic acids receiving a transposon insertion, for instance by selecting for expression of a selectable marker on the transposon, and screening said insertions for in-frame insertions. As described above, a full length reporter protein encoded by said transposon may be used to identify in-frame fusions, and the full length reporter protein may subsequently be removed by restriction digestion and ligation or cleaved to generate the internally fused complementing fragment. In-frame insertions may also be identified by sequencing across the junction between the inserted transposon DNA and the DNA encoding the interacting protein using a primer specific for transposon sequence and methods known in the art.

The present invention also encompasses a fusion protein encoded by such a nucleic acid and methods of making the same, for instance by recombinant expression of the nucleic acid in an appropriate cell from an appropriate promoter. Methods of purifying recombinantly expressed proteins are known in the art and are well within the capability of the skilled artisan. The invention also comprises a mixture comprising the fusion protein and at least a second fusion protein comprising the sequence of a second member of the binding pair fused to a second complementing fragment of the reporter protein, wherein the second complementing fragment does not produce detectable reporter protein activity in the absence of the first complementing fragment and the first complementing fragment is not present in said second fusion protein, and wherein the first and second complementing fragments do not have affinity for one another in the absence of said binding pair. In the mixture of the invention, the second complementing fragment may also be fused to the second binding member at an internal site of the second binding member. Only by binding of the first binding member to the second binding member is detectable reporter protein activity produced, via complementation of said first and second complementing fragments. Assays could also be designed using more than two complementing fragments, where none of the complementing fragments has affinity for another complimenting fragment, and wherein all complementing fragments are brought together to produce functional reporter protein activity by interacting members of a binding complex.

The mixtures or binding pairs of the present invention are useful for identifying compounds that modulate the binding of the binding pair. For instance, the present invention comprises a method of identifying a compound that modulates binding of a first protein to a second protein, comprising: (a) contacting a mixture comprising the binding pair with one or more test compounds; and (b) measuring reporter protein activity following said contacting, wherein increased or decreased reporter protein activity following said contacting indicates that a given test compound is a compound that modulates binding. In such methods, the first and second proteins may be a ligand and receptor, respectively, or any other protein pair where a modulating compound is desired. “Modulate” according to the present invention means that the modulating compound may either inhibit or facilitate or enhance the protein interaction of at least a first and second protein, wherein the interacting proteins are fused to complementing fragments of a reporter protein. Modulating compounds identified using the methods described herein are also included in the invention.

In the screening methods of the present invention, the fusion receptor protein containing the first complementing fragment may be expressed on the surface of a suitable host cell and the host cells aliquoted into the wells of a multiwell plate in the presence of a ligand fused to the complementing fragment such that many compounds may be screened simultaneously at a high throughput level. Similarly, other protein pairs may be expressed intracellularly and the host cells aliquoted into wells of a multiwell plate and exposed to each test compound. A vast collection of compounds could then be screened simultaneously for those that promote interaction of complementing fusion proteins, for instance those that promote dimerization or association of protein complexes, as well as for compounds that inhibit such interactions. Embodiments of the invention that employ GFP or a derivative thereof are particularly well suited for a wide variety of high throughput applications since 1) the fluorescence is an easy readout that does not require additional reagents, 2) one can screen for drugs that either cause two proteins to come together or which block a particular protein interaction, and 3) because the fusions are genetically encoded, they can be introduced into any possible cell type with a suitable promoter in hand. Depending on the reporter protein used and the conditions required for appropriate folding and measurement of activity, it may also be possible to provide either the first or second fusion protein binding member on a high density array.

The present inventors have surprisingly found that the binding pairs of the present invention may be selected that still retain physiological function despite the presence of the internally fused reporter protein fragment. Thus, the binding pairs of the present invention may also be used as a biosensor composition for detecting a change in concentration of an analyte having specificity for the binding pair The invention encompasses such biosensor compositions as described herein and methods of making and using the same to detect protein function and binding. The invention also encompasses single protein biosensors containing at least one internally fused complementing fragment and at least a second complementing fragment, wherein changes in protein conformation, for instance as a result of binding a ligand or analyte, are measured by detecting a positive or negative change in the level of reporter protein activity.

In the context of the present invention, a “biosensor” refers to a composition for the detection of an analyte that combines a binding pair component and a complementation component, wherein the binding pair interacts with or responds to the analyte, thereby leading to detectable changes in complementation and reporter protein activity. The binding pair may constitute two domains or regions of a single protein that come into close enough proximity upon protein folding, or in response to the binding of an analyte, such that changes in complementation or reporter protein activity based on analyte binding may be detected. The binding pair may also constitute two separate proteins or separate subunits of a multi-subunit protein complex, for instance a voltage-gated ion channel, such that changes in the concentration of the analyte lead to a change in binding interaction and a measurable change in reporter protein activity. Any analyte may be detected using the biosensors of the invention so long as the complementing fragments of a reporter protein may be genetically fused to one or more interacting proteins that interact with or respond to the presence of the analyte by changing conformation or binding affinity to one another. It has already been shown that complementation between proteins with GFP fragments fused to the ends can produce ligand-dependent changes in fluorescence (Demidov V V, et al. (2006) Fast complementation of split fluorescent protein triggered by DNA hybridization. Proc Natl Acad Sci USA 103:2052-6). The invention described here extends the realm of possible biosensors by facilitating complementation between proteins, or subunits, of a complex, or between domains of a single protein.

### EXAMPLES

### Example 1

### Construction of a Synthetic Tn5 Transposon

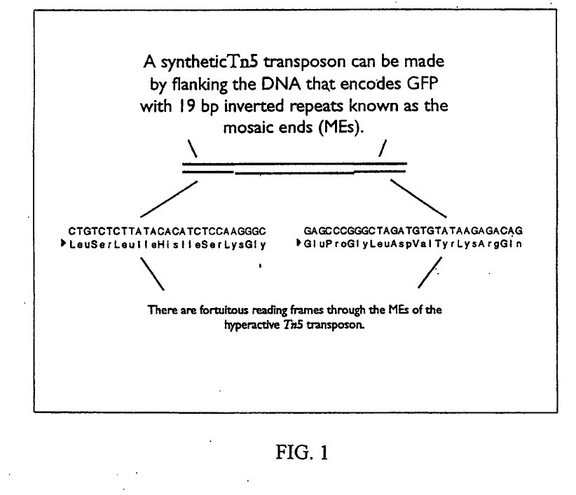

To create a transposon for generating random fusions to complementing fragments of a GFP reporter, we used the mosaic ends from a hyperactive Tn5 transposon. The mosaic ends are simply 19 base pair inverted repeats (SEQ ID Nos. 1 and 2) that can be placed on either side of any stretch of DNA to create a Tn5 transposon (see FIG. 1). These Tn5 ends are particularly suitable for creating fusion proteins in that there is an open reading frame that crosses the ends in either orientation.

We created a Tn5 transposon (pBonjovi) with PCR and standard subcloning techniques that carried the mosaic ends on either end of a segment of DNA that would, in combination with recombinant Tn5 transposase become inserted into a target plasmid. This is an in vitro reaction in which the transposase recognizes the mosaic ends of the transposon and inserts the transposon in a reasonably random fashion into any other DNA present in the reaction. We positioned the YFP variant Venus such that if the transposon is inserted into a sequence encoding another protein, in the correct orientation and relative reading frame, it will initially produce a fluorescent fusion protein that is truncated and has a yellow fluorescent protein attached to the C-terminus (See FIG. 2). The transposon was created by first amplifying the YFP/venus coding region with PCR primers that added the mosaic ends as well as additional restriction sites. These restriction sites were in turn used to introduce a Kanamycin resistance gene, isolated from the plasmid pBNJ24.6 (Sheridan D L, et al. (2002) A new way to rapidly create functional, fluorescent fusion proteins: random insertion of GFP with an in vitro transposition reaction. BMC Neurosci 3:7) and an additional YFP restriction fragment that contained the sequence encoding amino acids 159 to 238.

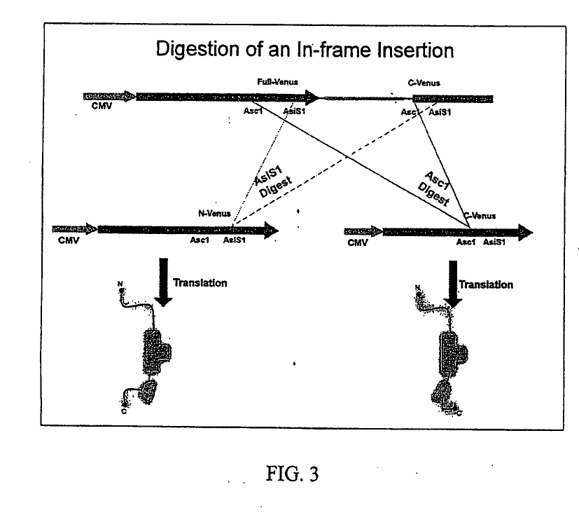

The transposon was created such that restriction digestion with either Asc I or AsiS I would leave either amino acids 1-158 of the fluorescent protein or amino acids 159-238. Then the sequence is re-ligated, to produce a complete fusion protein that has either the front or back half of Venus inserted internally in the sequence (see FIG. 3). This transposon approach has been pioneered in our laboratory, and is the most efficient way of inserting GFP and its derivatives into other proteins. Also, variations of GFP may also be readily substituted since the similarity in structure between the different fluorescent proteins is so conserved that this invention will work with all of the GFP derivatives.

### Example 2

### Isolation of YFP Complementing Fragments Fused Internally to Shaker Subunits

To test the process, we targeted the Shaker potassium channel. The Shaker potassium channel is a voltage-gated ion channel that is composed of 4 identical subunits. Our rationale was that if we created many different versions of the subunit, containing the two different fragments of the fluorescent protein, we could use pairwise expression of the different subunits to determine whether any complementation could occur between adjacent subunits.

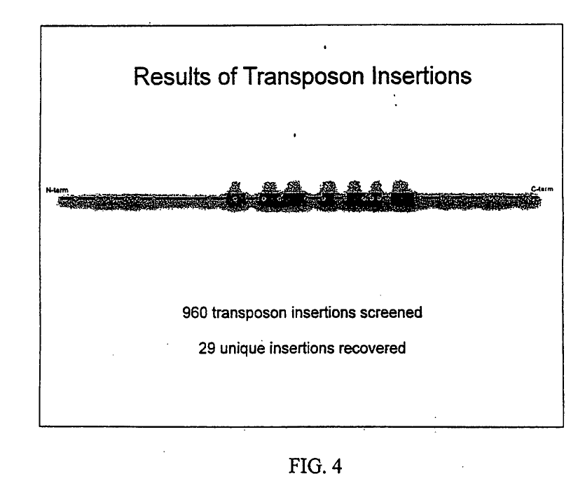

The sequence encoding the Shaker subunit was first moved as a restriction fragment into a small CMV expression plasmid. An in vitro reaction with the plasmid containing the Shaker subunit coding region, the transposon pBonjovi, and recombinant Tn5 transposase was used to insert the transposon sequence. Transformation of Top 10f′ E. coli with 1 ul of the 15 ul in vitro reaction produced greater than 3,000 colonies that displayed both the ampicillin resistance carried by the plasmid as well as the kanamycin resistance carried by the pBonjovi transposon. Miniprep DNA was isolated from 960 separate E. coli colonies, from the transformation. Transient transfection of 960 different wells of HEK 293 cells was used to identify the plasmids that encoded a fluorescent fusion protein. Each of the plasmids that produced a fluorescent signal was then sequenced using a primer complementary to the pBonjovi sequence. This made it possible to identify where the transposon was inserted into the Shaker coding sequence. There were several examples of duplicate insertions, and the duplicates were removed. In total, we recovered 29 unique transposon insertions in the subunit. We then used the different restriction enzymes to create 58 different subunits that contained either the first or second half of the fluorescent proteins. The 29 different insertions are shown in FIG. 4. We then co-expressed the different subunits, by transiently transfecting HEK 293 cells, to determine if any of the subunits could form a functional fluorophore.

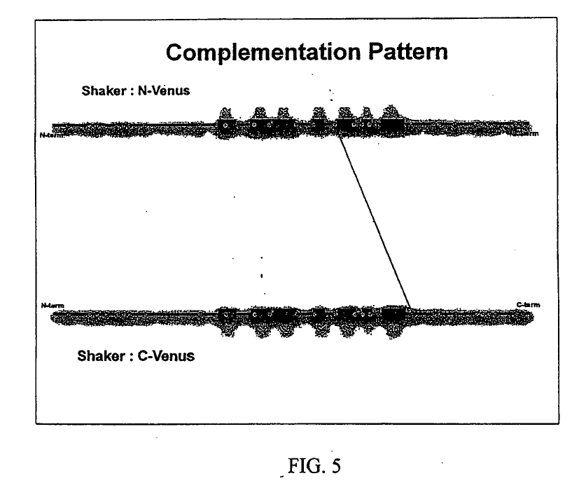

One strength of this combinatorial strategy is that one has the exponential power to find improbable combinations. In this case, 29 different fusions carrying the first half of YFP Venus were combined with 29 clones carrying the second half for a possible 841 combinations for complementation. In some cases, certain combinations of subunits could produce a fluorescent channel protein. For instance, FIG. 5 illustrates a combination of a subunit with the first fragment of Venus inserted between the 4th and 5th transmembrane domains and a subunit carrying the second fragment of Venus inserted just beneath the sixth transmembrane domain. This combination could produce a fluorescent protein.

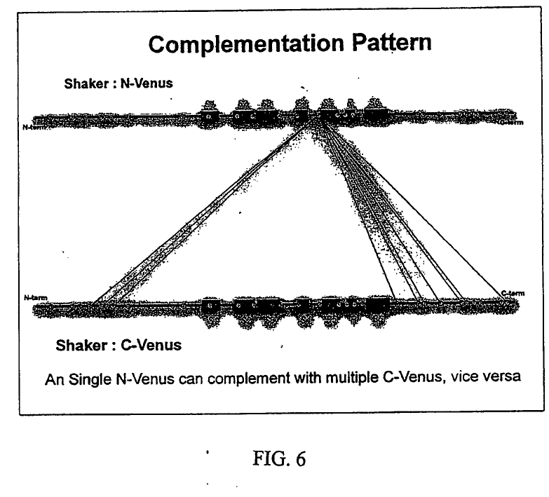

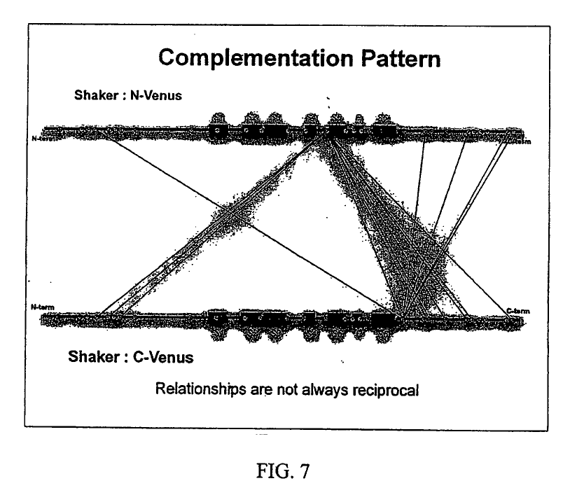

Further, it was often the case that a single insertion of one portion of the fluorescent protein could complement with other subunits carrying the second half in different locations (see FIG. 6). In other words, it was not always a one-to-one, specific pattern of complementation. Mapping these insertion sites onto the crystal structure shows that the insertions that work are often in flexible domains. Also, the complementation patterns were not always reciprocal. For example, the second half of the fluorescent protein just beneath the sixth transmembrane domain could complement with several different insertions of the front half, but not vice versa (see FIG. 7).

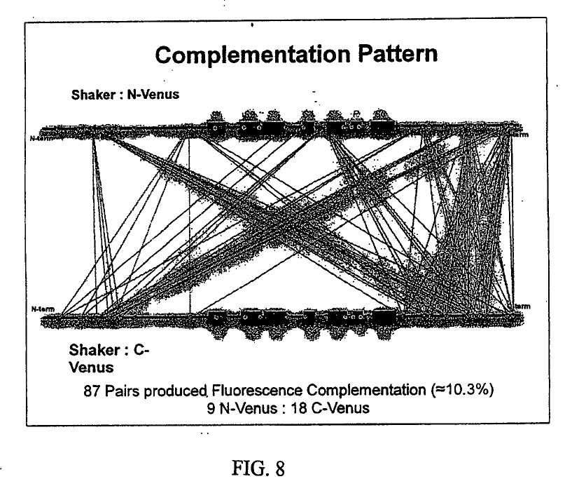

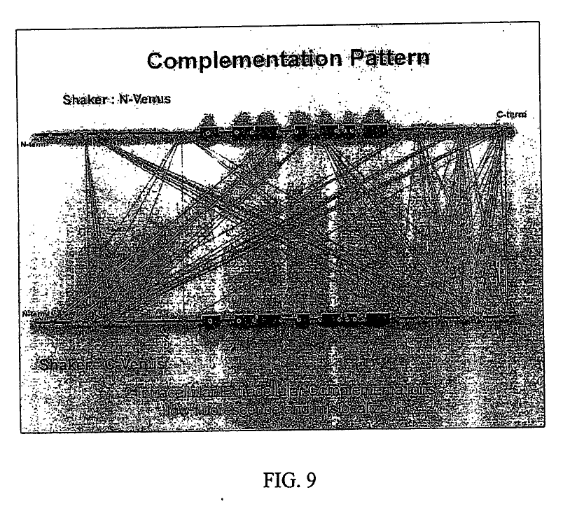

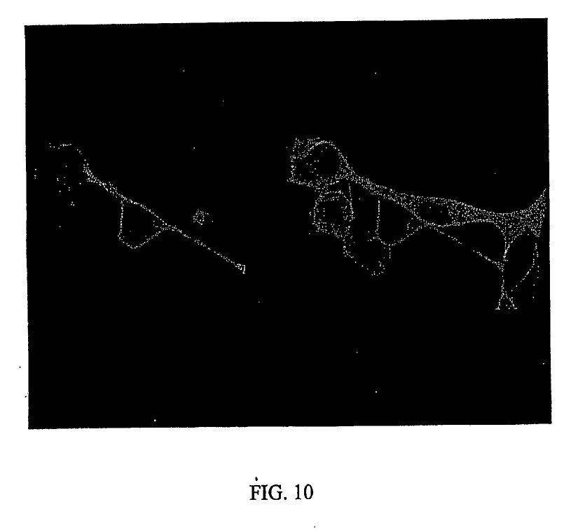

The total pattern of complementation observed is demonstrated in FIG. 8. We found that 87 pairs produced fluorescence complementation, or about 10.3% of the pairs that were screened. There were 9 insertion sites that contained the YFP fragment 1-158 that could complement with one or more insertion sites on another subunit the contained the YFP fragment 159-238. There were 18 insertion sites that contained the YFP fragment 159-238 that could complement with one or more insertion sites on a different subunit that contained the YFP fragment 1-158. The insertions that could produce fluorescence were distributed across the entire primary sequence of the subunit, showing that complementation can occur between fluorescent protein fragments that are quite far from the termini of the subunits. Two of the pairs were at positions that should not have produced fluorescence since they were at extra and intracellular sides of the channel respectively (FIG. 9). These two pairs gave only very low fluorescence apparently resulting from these fusions being retained within the endoplasmic reticulum. A surprising result was that the complementing pairs were targeted to the plasma membrane in HEK 293 cells much better than previously tagged channel proteins having full length GFPs (data not shown). FIG. 10 shows membrane fluorescence of shaker fusion complementing subunits (left) compared to the same cells expressing a membrane targeted red fluorescent protein.

We initially chose YFP Venus because it folds faster/better than the other GFP derivatives (Nagai et al., 2002, Nat. Biotechnol. 20: 87-90). We have since swapped in a variety of fluorescent proteins including CFP, Citrine, GFP and YFP, and all have worked.

### Example 3

### Measuring Voltage Dependent Changes Using Shaker Complementing Subunits

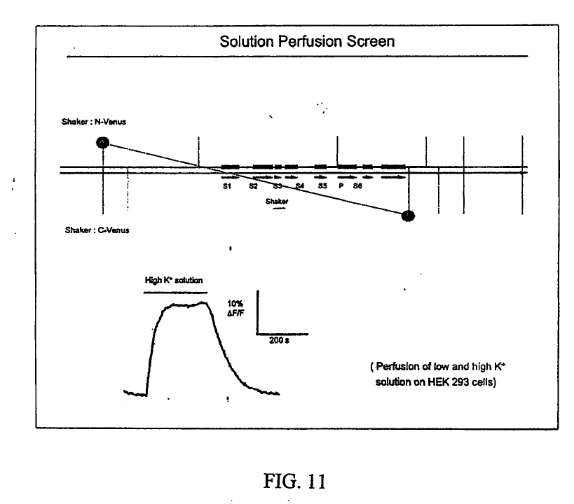

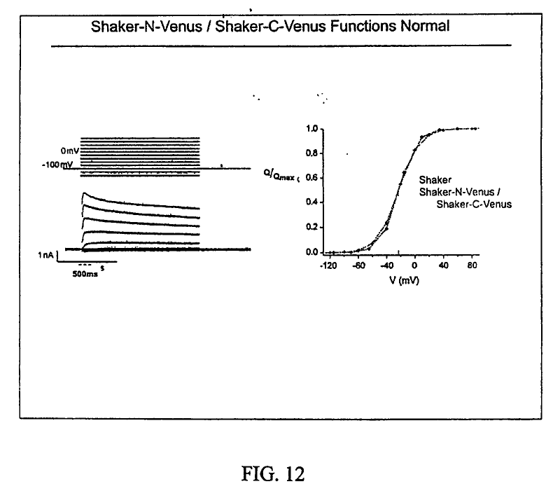

When the complementing pairs were screened for voltage dependent changes in fluorescence, two pairs of subunits were identified that produced changes in fluorescence of approximately 20% as a result of depolarization of the cells with high concentrations of extracellular potassium (see FIG. 11). Thus, interacting complementing subunits may also be used as new biosensors for detecting changes in extracellular potassium. Voltage versus gating measurements indicate that some pairs of the complementing subunits produce a normally functioning channel when expressed in HEK 293 cells, showing that complementing pairs may be isolated that retain the function of the interacting subunits (see FIG. 12).

The results of our work show that GFP fragments can be inserted deep in the structure of two interacting proteins and still complement one another to form a fluorophore. This means that the approach can be used with any set of interacting proteins in living cells. Using standard transposition reactions (Sheridan D L, et al., BMC Biotechnol (2004). 4,17: Sheridan D L, et al. BMC Neurosci (2002), 3,7) and the transposon we have described, a scientist skilled in molecular biology can rapidly place GFP fragments throughout two interacting proteins. Subsequent expression of the interacting proteins can then be used to identify the pairs that can complement. This enables investigators to search for unknown protein interactions, to optimize and visualize known protein interactions, or to design new kinds of biosensors. For example, many important signaling pathways in cells involve dimerization of receptors and effectors. This dimerization could be monitored through the complementation of GFP fragments placed within the two interacting proteins.

It is understood that the foregoing examples are merely illustrative of the present invention. Certain modifications of the articles and/or methods employed may be made and still achieve the objectives of the invention. Such modifications are contemplated as within the scope of the claimed invention.

All patents, patent applications, provisional patent applications and publications referred to or cited herein, are incorporated by reference in their entirety to the extent they are not inconsistent with the explicit teachings of the specification.

What is claimed:

1. A method of detecting interaction of at least a first and second protein or protein domain by complementation of at least two fragments of a reporter protein, wherein said at least two fragments of said reporter protein are genetically fused to said first and second proteins or protein domains, respectively, and at least one complementing fragment is fused at an internal position within one of said first or second interacting proteins or protein domains.

2. The method of claim 1, wherein each of said at least two complementing fragments is fused at an internal position within one of said first or second interacting proteins.

3. The method of claim 2, wherein each of said genetic fusions is constructed by transposon insertion.

4. The method of claim 3, wherein a selectable marker gene is removed from said transposon following insertion.

5. The method of claim 4, wherein both said first and second genetic fusions are generated using the same transposon following differential restriction digestion.

6. The method of claim 5, wherein said transposon comprises a sequence of elements according to the formula:
R1-F1-R2-S—R1-F2-R2
wherein R1 and R2 are first and second unique restriction sites, respectively;
F1 and F2 are complementing fragments of said reporter protein; and
S is a selectable marker gene.

7. The method of claim 6, wherein said selectable marker gene is a kanamycin resistance gene.

8. The method of claim 6, wherein R1 is an AscI restriction site.

9. The method of claim 6, wherein R2 is an AsiSI restriction site.

10. The method of claim 6, wherein F1 consists of amino acids 1 to 158 of a GFP derivative and F2 consists of amino acids 159-238 of a GFP derivative.

11. The method of claim 6, wherein said transposon is a Tn5 derivative.

12. A transposable complementation system for detecting protein interactions comprising at least one transposon encoding at least one complementing fragment of a reporter protein.

13. The transposable complementation system of claim 11 comprising at least two transposons, wherein each transposon encodes at least one complementing fragment of a reporter protein.

14. The transposable complementation system of claim 12 comprising a single transposon encoding at least a first and a second complementing fragment of a reporter protein.

15. The transposable complementation system of claim 14 further comprising a selectable marker gene.

16. The transposable complementation system of claim 15, wherein said first and second complementing fragments are separated by said selectable marker gene.

17. The transposable complementation system of claim 16, wherein said first complementing fragment is contained in an open reading frame encoding a functional reporter protein.

18. The transposable complementation system of claim 16, wherein said transposon comprises a sequence of elements according to the formula:
R1-F1-R2-S—R1-F2-R2
wherein R1 and R2 are first and second unique restriction sites, respectively;
F1 and F2 are nucleic acid sequences encoding complementing fragments of said reporter protein; and
S is a selectable marker gene.

19. The transposable complementation system of claim 12, wherein said at least one transposon is a Tn5 derivative.

20. The transposable complementation system of claim 12, wherein said reporter protein is green fluorescence protein (GFP) or a derivative thereof.

21. The transposable complementation system of claim 14, wherein said first complementing fragment is an amino terminal complementing fragment of YFP Venus.

22. The transposable complementation system of claim 21, wherein said amino terminal complementing fragment consists essentially of amino acids 1 to 158 of Venus.

23. The transposable complementation system of claim 14, wherein said second complementing fragment is a carboxyl terminal complementing fragment of YFP Venus.

24. The transposable complementation system of claim 23, wherein said amino terminal complementing fragment consists essentially of amino acids 159-238 of Venus.

25. The transposable complementation system of claim 18, wherein said selectable marker gene is a kanamycin resistance gene.

26. The transposable complementation system of claim 18, wherein R1 is an AscI restriction site.

27. The transposable complementation system of claim 18, wherein R2 is an AsiSI restriction site.

28. The transposable complementation system of claim 18, wherein F1 consists of amino acids 1 to 158 of GFP or a derivative thereof and F2 consists of amino acids 159-238 of GFP or a derivative thereof.

29. A kit comprising the transposable complementation system of claim 12, further comprising a transposase or transposase gene encoding a transposase that can catalyze transposition of said at least one transposon.

30. A kit comprising the transposable complementation system of claim 18, further comprising restriction enzymes R1 and R2.

31. A kit comprising the transposable complementation system of claim 12, further comprising instructions for isolating one or more transposon insertions in a target nucleic acid or group of target nucleic acids.

32. A method of detecting interaction of two or more protein domains using the transposable complementation system of claim 12, comprising:
(a) inserting randomly into one or more target nucleic acids one or more transposons encoding at least a first complementing fragment of a reporter protein;
(b) expressing said one or more target nucleic acids receiving said transposon insertions to produce one or more fusion proteins containing said first complementing fragment of said reporter protein;
(c) exposing said one or more fusion proteins containing said first complementing fragment of said reporter protein to one or more fusion proteins containing one or more other complementing fragments of said reporter protein; and
(d) detecting reporter protein activity;
wherein said first complementing fragment complements said one or more other complementing fragments to form a functional reporter protein, and wherein detectable activity of said reporter protein indicates interaction of two or more protein domains.

33. The method of claim 32, wherein said two or more protein domains are located in a single protein.

34. The method of claim 32, wherein said two or more protein domains are located in two or more subunits of a single protein complex.

35. The method of claim 32, wherein said two or more protein domains are located in two or more subunits of a dimeric or multimeric receptor complex.

36. The method of claim 32, wherein said two or more protein domains are located in two or more different proteins.

37. The method of claim 36, wherein said two or more protein domains are located in interacting ligand and receptor proteins, respectively.

38. The method of claim 36, wherein said two or more protein domains are located in different proteins that participate in a common signal transduction pathway.

39. The method of claim 32, wherein in step (c), said one or more fusion proteins containing said first complementing fragment of said reporter protein are exposed to one or more fusion proteins containing a second complementing fragment of said reporter protein; and
wherein said first complementing fragment complements said second complementing fragment to form a functional reporter protein, and wherein detectable activity of said reporter protein indicates interaction of two or more protein domains.

40. The method of claim 39, wherein said first and second complementing fragments are fragments of GFP or a derivative thereof.

41. The method of claim 40, wherein said first fragment consists essentially of amino acids 1 to 158 of YFP Venus and said second fragment consists essentially of amino acids 159 to 238 of YFP Venus.

42. The method of claim 39, wherein said one or more fusion proteins containing said second complementing fragment of said reporter protein are expressed from one or more nucleic acids comprising one or more transposons encoding said second complementing fragment.

43. The method of claim 42, wherein in step (a), target nucleic acids receiving said transposon insertions are isolated by selecting for expression of a selectable marker gene on said transposon.

44. The method of claim 43, wherein said selectable marker is an antibiotic resistance gene.

45. The method of claim 44, wherein said antibiotic resistance gene encodes kanamycin resistance.

46. The method of claim 43, wherein after step (a) but before step (b), said selectable marker genes on said transposon insertions are removed from each nucleic acid such that expression in step (b) generates fusion proteins that comprise said first complementing fragment at an internal position in each expressed protein.

47. The method of claim 46, wherein said selectable marker genes are removed by restriction digestion.

48. The method of claim 42, wherein said one or more nucleic acids comprising one or more transposons encoding said second complementing fragment were isolated by selecting for expression of a selectable marker gene on said transposon.

49. The method of claim 48, wherein said selectable marker is an antibiotic resistance gene.

50. The method of claim 49, wherein said antibiotic resistance gene encodes kanamycin resistance.

51. The method of claim 48, wherein said selectable marker genes on said transposon insertions containing said second complementing fragment were removed to generate nucleic acids encoding fusion proteins that comprise said second complementing fragment at an internal position in each expressed protein.

52. The method of claim 48, wherein said one or more transposons encoding at least a first complementing fragment of a reporter protein and said one or more transposons encoding said second complementing fragment are the same transposon.

53. The method of claim 52, wherein said transposon is cleaved differently following insertion to generate fusion proteins that comprise only said first complementing fragment or only said second complementing fragment, respectively.

54. The method of claim 53, wherein said transposon comprises a sequence of elements according to the formula:
R1-F1-R2-S—R1-F2-R2
wherein R1 is a restriction site for AscI;
R2 is a restriction site for AsiSI;
F1 encodes amino acids 1-158 of GFP or a derivative thereof;
F2 encodes amino acids 159-238 of GFP or a derivative thereof; and
S is a selectable marker gene.

55. The method of claim 54, wherein said transposon is a Tn5 derivative.

56. The method of claim 54, wherein said selectable marker gene encodes kanamycin resistance.

57. An isolated nucleic acid which encodes a fusion protein comprising the sequence of a first member of a binding pair fused to a first complementing fragment of a reporter protein, wherein said complementing fragment does not produce detectable reporter protein activity in the absence of at least one other complementing fragment that is not present in said fusion protein, and wherein said complementing fragment is fused to said binding member at an internal site of said binding member.

58. The nucleic acid of claim 57, wherein said complementing fragment is fused at an internal site that is at least about 5, at least about 10, at least about 20, at least about 30, at least about 40, at least about 50, at least about 75, at least about 100, at least about 150, at least about 250, or at least about 500 amino acids or more from either the amino or carboxyl terminus of said binding member.

59. The isolated nucleic acid of claim 57, wherein said reporter protein is GFP.

60. The nucleic acid of claim 59, wherein said fragment consists essentially of amino acids 1-158 of GFP.

61. The nucleic acid of claim 58, wherein said fragment consists essentially of amino acids 159-238 of GFP.

62. The fusion protein encoded by the nucleic acid of claim 57.

63. A mixture comprising the protein of claim 62 and a second fusion protein comprising the sequence of a second member of said binding pair fused to a second complementing fragment of said reporter protein, wherein said second complementing fragment does not produce detectable reporter protein activity in the absence of said first complementing fragment and said first complementing fragment is not present in said second fusion protein, and wherein said first and second complementing fragments do not have affinity for one another in the absence of said binding pair.

64. The mixture of claim 63, wherein said second complementing fragment is fused to said second binding member at an internal site of said second binding member.

65. The mixture of claim 63, wherein binding of said first binding member to said second binding member produces detectable reporter protein activity via complementation of said first and second complementing fragments.

66. A method of identifying a compound that modulates binding of a first protein to a second protein, comprising:
(a) contacting the mixture of claim 63 with one or more test compounds; and
(b) measuring reporter protein activity following said contacting,
wherein increased or decreased reporter protein activity following said contacting indicates that said test compound is a compound that modulates binding.

67. The method of claim 66, wherein said first and second protein are a ligand and receptor pair, respectively.

68. The method of claim 67, wherein said receptor protein is expressed on a cell surface.

69. A high-throughput screening procedure comprising the method of claim 66, wherein one of said first or second fusion proteins is provided on an array.

70. The method of claim 66, wherein said first and second fusion proteins are expressed intracellularly.

71. A biosensor comprising the mixture of claim 65, wherein a change in reporter protein activity is indicative of a change in the concentration of an analyte, said change in concentration of said analyte affecting binding of said first and second binding members.
